(19) United States
(12) Patent Application Publication
(10) Pub. No.: US 2006/0265028 A1
(43) Pub. Date: Nov. 23, 2006

(54) Hair growth

(75) Inventors: Jean-Marie Houle, Vancouver (CA); David W.C. Hunt, Surrey (CA)

Correspondence Address: MORRISON & FOERSTER LLP, 12531 HIGH BLUFF DRIVE, SUITE 100, SAN DIEGO, CA, 92130-2040, US

(73) Assignee: QLT INC., Vancouver (CA)

(21) Appl. No.: 10/554,310

(22) PCT Filed: Apr. 23, 2003
(86) PCT No.: PCT/CA03/00603

§ 371 Date: May 26, 2006

(51) Int. Cl. A61B 18/18; A61N 5/06

(52) U.S. Cl. 607/88; 606/9

(57) ABSTRACT

It has been discovered that photodynamic therapy (PDT) can stimulate an increase in hair count numbers and restore hair growth in areas of hair loss. A method according to the present invention comprises: a) administering an effective and/or sufficient amount of a photosensitizer to the target skin; b) irradiating the target skin with energy comprising one or more wavelength capable of activating said photosensitizer for a time period sufficient to activate the photosensitizer, wherein there is an increase in hair count numbers in the treated area. In one aspect of the present invention, there is a 2% or more increase in the number of terminal hairs within 3 months.

### FIELD OF THE INVENTION

This invention relates to the use of photodynamic therapy to stimulate hair growth. In particular, the present invention relates to the use of photosensitizers and PDT for treating conditions relating to hair loss, such as androgenetic alopecia and alopecia areata, is described. The present invention further relates to a method of photodynamic therapy that causes an increase in the level of pro-inflammatory cytokines thereby causing hair growth and to a method of determining the increase in hair growth.

### BACKGROUND OF THE INVENTION

Alopecia is the general term referring to any disease or condition involving hair loss. There are several different types of hair loss, such as androgenetic alopecia (AGA; see Sawaya, M. E. Seminars in Cutaneous Medicine and Surgery 17(4):276-283, 1998), alopecia areata (AA; see Fiedler & Alaiti, Dermatologic Clinics 14(4): 733-738, 1996), as well as chemotherapy and rug-induced alopecia Androgenetic alopecia (AGA) is by far the most common type of alopecia. AGA is a patterned, progressive loss of an excessive amount of hair from the scalp. Significant AGA occurs in 50% of men by the age of fifty and 50% of women by the age of sixty. AGA is believed to be a result of both genetic predisposition and the presence of a sufficient level of circulating androgens. It is thought that the enzyme 5-alpha reductase present in dermal papilla cells converts testosterone to dihydrotestosterone (DHT). DHT binds to androgen receptors, also localized in the dermal papilla cells, triggering changes in the hair follicle that result in (1) shortening of the anagen or growth phase of the hair cycle, (2) development of a latent phase in the hair cycle following shedding of the telogen hair, and (3) follicular miniaturization process that reduces the caliber of the anagen hairs produced. It is thought that differential expression of 5-alpha reductase and/or androgen receptors in various types of hair follicles accounts for patterned hair growth and loss.

Currently approved treatments for AGA include minoxidil (Rogaine™), an anti-hypertensive drug for which the mechanism of action in promoting hair growth is unknown. Minoxidil must be applied topically on a twice daily basis, and is therefore somewhat inconvenient to use. Studies have shown that 2% Minoxidil can provide an increase in the numbers of terminal hairs after 4-12 months (De Villez et al, Journal of the American Academy of Dermatology, Vol. 16, No. 3, Part 2 (March 1987) 669-672). However, this benefit disappears over time or once the treatment is stopped. Another drug used in the treatment of AGA is finasteride (Propecia™), a selective inhibitor of the type 2 isoenzyme 5-alpha reductase. This treatment has marginal efficacy, requires daily oral administration and can have anti-androgenic side effects such as alteration of libido. Hair transplants and scalp reduction are also performed on patients with hair loss associated with AGA. These procedures are too expensive or time-consuming for many people. In addition, many people are put off by the surgical nature of the treatment. Photochemotherapy therapy has been proposed as a treatment for alopecia areata (AA). The proposed therapy, using psoralen and high energy UVA (PUVA) treatment, has met with very limited success and its effectiveness for AA is in doubt (Lebwohl, M. Lancet 349:222-223, 1997). Side effects of PUVA treatment such as nausea, pigmentary changes, risk of skin cancer formation, and cataracts have been reported (Fiedler & Alaiti, Dermatologic Clinics 14(4): 733-738, 1996). Antioxidants have been used to ameliorate the side-effects of PUVA therapy (Ptapenko & Kyagova, Membr. Cell Biol. 12(2): 269-278, 1998). The use of 2% khellin, a compound with a chemical structure that resembles psoralen, and UVA for alopecia areata was found to be successful in 5 of the 10 patients tested (Orasa et al. Int. J. Dermatol. 32(9): 690, 1993). Since Khellin did not cause phototoxicity, the authors have suggested its use as an alternative to psoralen. Hematoporphyrin and high energy UVA has been used in a very limited study by Monfrcola et al. (Photodermatology 4:305-306, 1987). Two patients were treated with topical hematoporphyrin (0.5%, HP) and UVA irradiation with three times a week for eight weeks. In the first week of treatment there was significant erythema and mild scaling followed by hyperpigmentation in the HP treated sites. Side effects included unpleasant reddish skin coloration for several hours and sometimes burning sensations during the irradiation phase. The authors point out that severe phototoxic reactions could occur with the use of HP concentrations greater than 1%. They also state that more work is needed before this approach can be subject to routine clinical use.

There exists a need for an effective, non-surgical procedure that results in a rapid increase in the numbers of terminal hairs but has minimal side effects.

Photodynamic therapy (PDT) is a minimally invasive two-step medical procedure that uses light-activated drugs called photosensitizers to treat a range of diseases. First, a photosensitizer is administered and, once it has permeated the target tissue, the photosensitizer is then activated by exposure to a dose of light at a particular wavelength. Photodynamic therapies have been approved for a number of indications including the treatment of non-small cell lung cancer (Photofrin™), age-related macular degeneration (Visudyne™), actinic keratosis (Metvix™, Levulan™), and basal cell carcinoma (Metvix™).

It has been suggested that PDT can be utilized for the removal of unwanted hair in human subjects. Briefly the treatment involves a topical application of a photosensitizer on a selected area of the skin, a period for absorption of the photosensitizer, followed by a pulse or continuous irradiation or vibration of the area. The process involves inactivating or destroying the hair follicles or destroying the tissue feeding the hair follicles (see U.S. Pat. Nos. 5,669,916; 5,871,480; WO 97/32046).

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

It has been discovered that photodynamic therapy (PDT) can stimulate an increase in hair count numbers and restore hair growth in areas of hair loss. One aspect of the present invention comprises:
(a) administering an effective and/or sufficient amount of a photosensitizer to the target skin,
(b) irradiating the target skin with light comprising one or more wavelength capable of activating said photosensitizer for a time period sufficient to activate the photosensitizer; and optionally
(c) repeating (a) and (b)
   wherein the there is an increase in hair count numbers in the treated area.

One aspect of the present invention relates to a method of treatment with PDT wherein there is a 2% or more increase in the number of terminal hairs within 3 months. Terminal hairs are long hairs that are produced by follicles with sebaceous glands. They are in contrast to vellus hairs, which are short hairs, often only a centimeter or two long, that contain little or no pigment. The follicles that produce vellus hairs do not have sebaceous glands and do not produce any other kind of hairs. Terminal hairs also differ from Lanugo hair, which develops on an unborn baby.

The normal progression of conditions such as AGA is for a gradual decrease in the number of terminal hairs over time. The terminal hairs may also gradually become thinner and shorter until they look like vellus hairs. It is surprising, therefore, that the current method can actually increase the number of hairs after 3 months. In addition to treatment of hair loss, the method of the present invention may be used for the stimulation of hair growth in areas not recognized as experiencing hair loss.

As used herein, the term “hair growth” refers to an increase in number of terminal hairs present. Terminal hair counts can be conducted in a number of ways. For example, the terminal hair can be counted by trained and validated technicians who perform a computer-assisted count on macrophotographs. In brief, a target area on the scalp is chosen, the hair clipped and the scalp permanently marked with a single dot tattoo in the center in order to facilitate the exact positioning at each subsequent photo session. The macrophotography is performed using a preset camera with a macro lens and a stand that provides a constant reproduction ratio and electronic flashes that reproducibly illuminate the area to photograph. The images are taken in triplicate, centering the camera using the tattoo and the color slide films are processed at a central facility. The quality of the images is assessed and large transparencies are made of the best images. The terminal hairs on the target circle of the transparencies are then counted by the trained technicians.

### DETAILED DESCRIPTION

The present invention may be used with any subject capable of hair growth. Preferably, the invention is applied to skin tissue exhibiting, or suspected of, hair growth reduction or hair loss. Preferred subjects include mammals, with human subjects being particularly preferred. The present invention is useful for treating subjects, particularly humans, suffering from AGA.

While not wishing to be bound by theory, it is believed that the present method stimulates an increase in tissue levels of one or more specific growth factors and/or cytokines in the treated area. These factors then directly, or through other biochemical signaling pathways stimulate resting hair follicles to enter the anagen (growth) phase. In PDT-mediated hair growth it is believed that pro-inflammatory cytokines, such as interleukin-1-alpha, interleukin-1-beta, or granulocyte-macrophage colony stimulating factor (GM-CSF), play a role in inducing hair, particularly terminal hair, growth as described herein. Cells capable of producing such cytokines include macrophages, keratinocytes, dermal fibroblasts, dermal papilla cells, and T-cells. The invention also provides for the use of such increases in the treatment of other conditions.

Pro-inflammatory cytokines, such as IL-1 and GM-CSF, are known to have a wide range of effects within tissues. These actions may include stimulating the production of various biochemical mediators, up-regulating the expression of specific cell surface receptors and triggering the activation and tissue infiltration of pro-inflammatory cell types including neutrophils and macrophages. However, it is surprising that an increase in IL-1 can cause an increase in the number of terminal hairs since there is a body of evidence that suggests IL-1 induces hair loss rather than hair growth (see, for example, Dermatology 1995;191:273-275 Hoffmann et al; Eur J Dermatol 1998;8:475-7 Hoffmann et al; Lymphokine & Cytokine Research Vol.12, Number 4, 1993 Harmon et al).

Therefore, the present invention also relates to a method of increasing the levels of pro-inflammatory cytokines in the tissues of the skin by PDT. In particular, to a method of causing an increase in the number of terminal hairs by increasing the levels of pro-inflammatory cytokines in the target area by treating the area with photodynamic therapy. The present method preferably causes an increase in granulocyte-macrophage colony stimulating factor, interleukin-1-β (IL-1 -β), and/or interleukin-1-α (IL-1-α).

In one aspect, the present method provides at least 2% increase in the numbers of terminal hairs within 3 months of the PDT treatment. Preferably, the present invention provides a 3% or more increase, more preferably a 4% or more increase, in the numbers of terminal hairs within 3 months. The numbers of terminal hairs on a particular subject can be assessed by the validated method “Photographic Documentation of Hair Growth in Androgenetic Alopecia” (D. Canfield, Dermatologic Clinics, Vol. 14 No. 4 (October 1996) 713-721).

The present method can comprise:
(a) assessing the numbers of terminal hairs according to the above mentioned method;
(b) administering an effective amount of a photosensitizer;
(c) irradiating the target skin with activation energy comprising one or more wavelength capable of activating said photosensitizer for a time period sufficient to activate the photosensitizer; and
(d) optionally repeating (b) and (c);
(e) assessing the numbers of terminal hairs according to the above mentioned method;

wherein there is at least a 2%, preferably at least a 3%, more preferably at least a 4% increase in the numbers of terminal hairs as assessed within 3 months.

The present invention also relates to a method of determining the increase in hair growth in a subject's skin exhibiting hair growth reduction or hair loss. The method comprises:
a) administering a photosensitizer to the skin;
b) irradiating said skin with electromagnetic energy containing a wavelength absorbed by said photosensitizer to activate it; and
c) measuring the increase in hair growth,

wherein an increase in hair growth in comparison to skin that has not been treated with both a) and b) can be determined. It is preferred that skin that has not been treated has not been administered said photosensitizer and/or has not been irradiated.

Preferably the photosensitizer is selected from those which absorb radiation in the range 400 nm to 800 nm. Preferably the photosensitizer is administered by topical application. Preferably the electromagnetic energy is visible light.

The increase in hair growth can be measured by counting the number of terminal hairs, measuring hair weight, measuring hair density, and/or measuring hair shaft diameter. Preferably, the increase in hair growth is measured by counting the number of terminal hairs as described above.

Any suitable photosensitizing agent or mixture of agents may be used herein. Those which can be activated by visible light are preferred. Generally, these will absorb radiation in the range of from about 380 nm to about 900 nm. Preferred are those which absorb radiation in the range 400 nm to 800 nm. Those that absorb radiation in the range of from 600 nm to 750 nm are more preferred. Preferably, the photosensitizer is nontoxic to humans or is capable of being formulated in a nontoxic composition. Preferably, the chemical compound in its photodegraded form is also nontoxic. A photosensitizer may be defined as a substance that absorbs electromagnetic radiation, most commonly in the visible spectrum, and releases it as another for of energy, most commonly as reactive oxygen species and/or as thermal energy.

A listing of photosensitive chemicals may be found in Kreimer-Birnbaum, Sem. Hematol. 26:157-73, 1989 (incorporated herein by reference) and in Redmond and Gamlin, Photochem. Photobiol. 70 (4): 391-475 (1999). The invention may be practiced with a variety of synthetic and naturally occurring photosensitizers, including, but not limited to, pro-drugs such as the pro-porphyrin 5-aminolevulinic acid (ALA) and derivatives thereof such as aminolevulinic acid esters, porphyrins and porphyrin derivatives e.g. chlorins, bacteriochlorins, isobacteriochlorins, phthalocyanine and naphthalocyanines and other tetra- and poly-macrocyclic compounds, and related compounds (e.g. pyropheophorbides, sapphyrins and texaphyrins) and metal complexes such as, but not limited to, tin, aluminum, zinc, lutetium, tin ethyl etiopurpurin (SnET2). Tetrahydrochlorins, purpurins, porphycenes, and phenothiaziniums are also within the scope of the invention. Some examples of suitable compounds include, but are not limited to, those described in U.S. Pat. Nos. 6,462,192; 6,444,194; 6,376,483; WO-A-03/028628; WO-A-03/028629; WO-A-02/096417; and WO-A-02/096366, all of which are herein incorporated by reference.

Preferably the photosensitizers herein are selected from pro-porphyrins, porphyrins, and mixtures thereof. Some examples include aminolevulinic acid such as Levulan™, aminolevulinic acid esters such as described in WO-A-02/10120 and available as Metvix™, Hexvix™ and Benzvix™, di-hydro or tetra-hydro porphyrins such as described in described in EP-A-337,601 or WO-A-01/66550 and available as Foscan™ (temoporfin), porfimer sodium (available as Photofrin™), Visudyne™, benzoporphyrin derivatives (which are described in more detail below), and mixtures thereof.

In preferred embodiments of the invention, the photosensitizer is selected from a particularly potent group of photosensitizers known as green porphyrins, which are described in detail in U.S. Pat. No. 5,171,749 (incorporated herein by reference). The term “green porphyrins” refers to porphyrin derivatives obtained by reacting a porphyrin nucleus with an alkyne in a Diels-Alder type reaction to obtain a mono-hydrobenzoporphyrin. Such resultant macropyrrolic compounds are called benzoporphyrin derivatives (BPDs), which is a synthetic chlorin-like porphyrin with various structural analogues, as shown in U.S. Pat. No. 5,171,749. Typically, green porphyrins are selected from a group of tetrapyrrolic porphyrin derivatives obtained by Diels-Alder reactions of acetylene derivatives with protoporphyrin under conditions that promote reaction at only one of the two available conjugated, nonaromatic diene structures present in the protoporphyrin-IX ring systems (rings A and B). Metallated forms of a Gp, in which a metal cation replaces one or two hydrogens in the center of the ring system, may also be used in the practice of the invention. The preparation of the green porphyrin compounds useful in this invention is described in detail in U.S. Pat. No. 5,095,030 (hereby incorporated by reference).

Preferably, the BPD is a benzoporphyrin derivative diester di-acid (BPD-DA), mono-acid ring A (BPD-MA), mono-acid ring B (BPD-MB), or mixtures thereof. These compounds absorb light at about 692 nm wavelength and have improved tissue penetration properties. The compounds of formulas BPD-MA and BPD-MB may be homogeneous, in which only the C ring carbalkoxyethyl or only the D ring carbalkoxyethyl would be hydrolyzed, or may be mixtures of the C and D ring substituent hydrolyzates. A number of other BPD B-ring derivatives may also be used in the present methods. These derivatives have the following general formula:

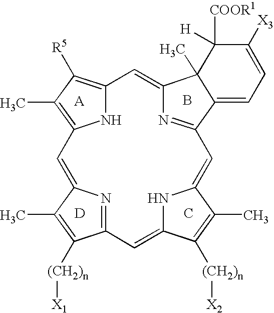

wherein; R⁵ is vinyl, R¹ and R⁶ are methyl, and n is 2. X₁, X₂, and X₃ are listed in the tables below:

TABLE 1
Hydrophilic BPD B-ring analogs

| Drug | X₁ | X₂ | X₃ |
|---|---|---|---|
| QLT0061 | COOH | COOH | COOH |
| QLT0077 | CONH(CH₂)₂N⁺(CH₃)₃I⁻ | CONH(CH₂)₂N⁺(CH₃)₃I⁻ | COOCH₃ |
| QLT0079 | CONH(CH₂)₂N⁺(CH₃)₂((CH₂)₃CH₃ | CONH(CH₂)₂N⁺(CH₃)₂((CH₂)₃CH₃) | COOCH₃ |
| QLT0086 | CONHCH(COOH)CH₂COOH | CONHCH(COOH)CH₂COOH | COOCH₃ |
| QLT0092 | CONH(CH₂)₂NH(CH₃)₂ | CONH(CH₂)₂NH(CH₃)₂ | COOCH₃ |
| | CF₃COO⁻ | CF₃COO— | |
| QLT0094 | CONHCH₂COOH | CONHCH₂COOH | CONHCH₂COOH |

TABLE 2
Lipophilic BFD B-ring analogs

| Drug | X1 | X2 | X3 |
|---|---|---|---|
| QLT0060 | CO(O(CH₂)₂)OH | CO(O(CH₂)₂)0H | COOCH₃ |
| QLT0069 | COOCH₃ | COOCH₃ | COOH |
| QLT0078 | CO(O(CH₂)₂)₂OH | CO(O(CH₂)₂)₂OH | COOCH₃ |
| QLT0080 | CO(O(CH₂)₂)₃OH | CO(O(CH₂)₂)₃OH | COOCH₃ |
| QLT0081 | CO(O(CH₂)₂)₂OCH₃ | CO(O(CH₂)₂)₂OCH₃ | CO(O(CH₂)₂)₂OCH₃ |
| QLT0082 | CO(O(CH₂)₂)₂OH | CO(O(CH₂)₂)₂OH | CO(O(CH₂)₂)₂OH |
| QLT0083 | CO(O(CH₂)₂)₃OH | CO(O(CH₂)₂)₃OH | CO(O(CH₂)₂)₃OH |
| QLT0087 | CO(O(CH₂)₂)₄OH | CO(O(CH₂)₂)₄OH | COOCH₃ |
| QLT0088 | COOCH₃ | COOCH₃ | CONH(C₆H₄)(C₅H₁₀N) |
| QLT0090 | CO(O(CH₂)₂)₅OH | CO(O(CH₂)₂)₅OH | COOCH₃ |
| QLT0093 | CO(O(CH₂)₂)₅OH | CO(O(CH₂)₂)₅OH | CO(O(CH₂)₂)₅OH |

Preferred photosensitizers are the benzoporphyrin derivative mono-acid (BPD-MA), QLT0074 (as set forth in U.S. Pat. No. 5,929,105 referred to therein as A-EA6) and B3 (as set forth in U.S. Pat. No. 5,990,149). Most preferably the photosensitizer is QLT0074 which has the structure:

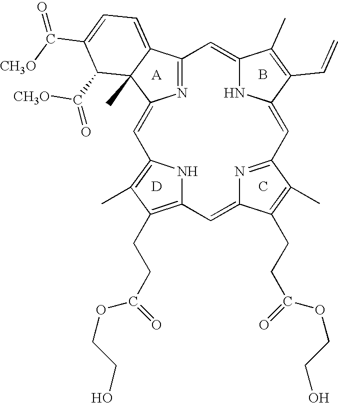

Additionally, the photosensitizers used in the invention may be conjugated to various ligands to facilitate targeting. These ligands include receptor-specific ligands as well as immunoglobulins and fragments thereof Preferred ligands include antibodies in general and monoclonal antibodies, as well as immunologically reactive fragments of both.

Dimeric forms of the green porphyrin and dimeric or multimeric forms of green porphyrin/porphyrin combinations can be used. The dimers and oligomeric compounds of the invention can be prepared using reactions analogous to those for dimerization and oligomerization of porphyrins per se. The green porphyrins or green porphyrin/porphyrin linkages can be made directly, or porphyrins may be coupled, followed by a Diels-Alder reaction of either or both terminal porphyrins to convert them to the corresponding green porphyrins. Combinations of two or more photosensitizers may also be used in the practice of the invention.

In addition to the above mentioned preferred photosensitizing agents, additional examples of photosensitizers useful in the invention include, but are not limited to, green porphyrins disclosed in U.S. Pat. Nos. 5,283,255, 4,920,143, 4,883,790, 5,095,030, and 5,171,749; and green porphyrin derivatives, discussed in U.S. Pat. Nos. 5,880,145 and 5,990,149. Several structures of typical green porphyrins are shown in the above cited patents, which also provide details for the production of the compounds.

A preferred photosensitizer for use in the present invention will satisfy the following general criteria: 1) it is capable of entry into the target hair follicles and/or the surrounding tissues and cells; and 2) irradiation, preferably with light (and more preferably with visible light), results in the stimulation of and/or restoration of hair growth.

In one embodiment, the methods of the invention are used to stimulate and/or restore hair growth after initial diagnosis. In another embodiment, the methods of the invention follow other treatments for alopecia, including PDT, as a form of maintenance therapy to prevent appreciable hair loss and/or maintain hair growth. The latter may be used to prevent or inhibit the re-occurrence of alopecia.

The present invention further relates to a method for causing an increase in the number of terminal hairs comprising administering photodynamic therapy to the area in which an increase in the number of terminal hairs is desired and administering at least one secondary treatment that causes an increase in the number of terminal hairs within the treatment area, wherein the secondary treatment is not photodynamic therapy. The non-photodynamic treatment can be any suitable regimen but is preferably one that increases terminal hair numbers via a different method of action from PDT treatment. For example, a local treatment or a systemic treatment. Preferably, the secondary treatment is selected from 5-alpha reductase inhibitors, minoxidil, hair transplantation, scalp reduction, and combinations thereof. More preferably, the secondary treatment is selected from 5-alpha reductase inhibitors, minoxidil, and combinations thereof. For example, Rogaine™ or Propecia™ maybe utilised in combination with PDT treatment(s).

One preferred method herein comprises:
a) topically administering photosensitizer to the target tissue,
b) irradiating the target tissue with radiation of a wavelength appropriate to activate the photosensitizer,
c) administering at least one, non-photodynamic, treatment that causes an increase in the number of terminal hairs within the treatment area.

The non-photodynamic treatment can be administered at any suitable time, before, concurrently or after the PDT. It is preferred that the non-photodynamic treatment is selected from 5-alpha reductase inhibitors, minoxidil, and combinations thereof

If the non-photodynamic treatment is minoxidil it is preferably used as a topical solution. Preferably the solution is administered from 1 to 4 times daily, more preferably twice daily. The solution can be any suitable strength but is preferably from about 1% to about 10%, more preferably about 2% or about 5%.

If the non-photodynamic treatment is 5-alpha reductase inhibitors it is preferably administered orally. Preferred 5-alpha reductase inhibitor is finasteride. Finasteride is preferably administered as a 1 mg oral table and is preferably taken once a day.

The methods of the invention can be used to stimulate hair growth in any situation in which additional hair growth is desired. In particular, the methods of the invention will be useful when the subject has experienced loss of hair associated with a variety of conditions, including, but not limited to the following: anagen effluvium, drug-induced alopecia, radiotherapy, poisoning, diffuse alopecia areata, alopecia areata, loose anagen syndrome, postoperative occipital alopecia, syphilis, traction alopecia, tricholtillomania tinea capitis, telogen effluvium, telogen gravidarum, chronic telogen effluvium, early androgenentic alopecia, iron deficiency, malnutrition/malabsorption, hypothyroidism, hyperthyroidism, systemic lupus erythematosus, chronic renal failure, hepatic failure, advanced malignancy, viral or bacterial infection, and androgenetic alopecia. In particular, the methods of the invention are useful for restoration of hair loss in androgenetic alopecia, drug-induced alopecia (for example following chemotherapy treatment for cancer), and hair loss due to radiation treatment.

The photosensitizers of the invention may be formulated into a variety of compositions. These compositions may comprise any component that is suitable for the intended purpose, such as conventional delivery vehicles and excipients including isotonising agents, pH regulators, solvents, solubilizers, dyes, gelling agents and thickeners and buffers and combinations thereof. Pharmaceutical formulations suitable for use with the instant photosensitizers can be found, for instance, in Remington's Pharmaceutical Sciences. Preferred formulations herein comprise pharmaceutical excipients or carriers capable of directing the photosensitizer to the area of hair growth reduction or hair loss. Suitable excipients for use with photosensitizers include water, saline, dextrose, glycerol and the like.

Typically, the photosensitizer is formulated by mixing it, at an appropriate temperature, e.g., at ambient temperatures, and at appropriate pHs, and the desired degree of purity, with one or more physiologically acceptable carriers, i.e., carriers that are nontoxic at the dosages and concentrations employed. Generally, the pH of the formulation depends mainly on the particular use, and concentration of photosensitizer, but preferably ranges anywhere from about 3 to about 8. Preferably, the photosensitizer is maintained at a pH in the physiological range (e.g., about 6.5 to about 7.5). The presence of salts is not necessary, and, therefore the formulation preferably is not an electrolyte solution.

The formulations herein preferably comprise a skin-penetration enhancer. Any skin-penetration enhancer suitable for aiding the delivery of the photosensitizing agent can be used herein. A list of skin-penetration enhancers can be found in “Pharmaceutical Skin Penetration Enhancement” (1993) Walters, K. A., ed.; Hadgraft, J., ed—New York, N.Y. Marcel Dekker and in “Skin Penetration Enhancers cited in the Technical Literature” Osbourne, D. W. Pharmaceutical Technology, November 1997, pp 59-65, both of which are incorporated herein by reference. Preferred for use in the formulations herein are hydrophobic skin-penetration enhancers.

Preferred skin-penetration enhancers are selected from glycol ethers, fatty acids, fatty acid esters, glycol esters, glycerides, azones, polysorbates, alcohols, dimethylsulfoxide, and mixtures thereof. Preferred skin-penetration enhancers for use herein include, but are not limited to, diethylene glycol monoethyl ether (Transcutol®), Oleyl alcohol, Oleic acid, Azone (Laurocapram or 1-n-Dodecyl azacycloheptan-2-one), Propylene glycol mono- and diesters of fats and fatty acids (e.g. propylene glycol monocaprylate, propylene glycol monolaurate), Triglycerides and lipids (e.g. linoleic acid), Macrogolglycerides or Polyethylene glycol glycerides and fatty esters (e.g. stearoyl macrogolglycerides, oleoyl macrogolglycerides, lauroyl macrogolglycerides, Oleyl macrogol-6-glycerides, Lauroyl macrogol-6 glycerides), Glycerides and fatty acid esters of polyethylene glycol (e.g. caprylocaproyl macrogolglycerides, capryl-caproyl macrogolglycerides, oleoyl macrogol glycerides), Polyoxyl 40 Hydrogenated Castor Oil (Cremophor RH 40), Polysorbate 80 (Tween 80), Dodecylazacycloheptanone, SEPA® such as described in U.S. Pat. No. 4,861,764 (e.g. 2-n-nonyl-1,3-dioxolane), and mixtures thereof. More preferred is diethylene glycol monoethyl ether (available from Gattefosse under the tradename Transcutol).

It is preferred that the formulations comprise from about 0.1% to about 99%, preferably from about 0.1% to about 90%, more preferably from about 5% to about 90%, even more preferably from about 15% to about 75%, by weight of skin penetration enhancer.

It is preferred that the ratio of photosensitizer to skin-penetration enhancer is from about 1:20 to about 1:10000, more preferably from about 1:60 to 1:300, on the basis of percentages by weight of total composition.

It is preferred that the photosensitizer is solubilised, especially when the photosensitizer is hydrophobic. One method of solubilising certain photosensitizers, including green porphyrins, is by formulation in liposomes. An alternative may be to solubilise the photosensitizer in cyclodextrins or cyclodextrin derivatives. Preferred are partially etherified cyclodextrin, the ether substituents of which are hydroxyethyl, hydroxypropyl or dihydroxypropyl groups. However, appropriate cyclodextrins should be of a size and conformation appropriate for use with the photosensitizing agents disclosed herein.

Other methods suitable for solubilising certain photosensitizers include the use of a solvent acceptable for use in the treatment of skin tissues and cells such as, but are not limited to, DMSO (dimethylsulfoxide), polyethylene glycol (PEG) or any other solvent. It is preferred that the formulations herein comprise a solubilizer. Some solubilizers are also penetration enhancers and it is preferred that the formulations herein comprise a penetration enhancer that is also a solubilizer for the photosensitizer. Preferably the solubilizer is selected from glycol ethers, polyethylene glycol, polyethylene glycol derivatives, propylene glycol, propylene glycol derivatives, polysorbates (e.g. Tween™), fatty alcohols, aromatic alcohols, propylene glycol, glycerols, oils, surfactants, glucosides, and mixtures thereof. More preferably the solubilizer is selected from diethylene glycol monoethyl ether (Transcutol®), polyethylene glycol of average molecular weight from 100 to 5000, triethylene glycol, tetraethylene glycol, pentaethylene glycol, hexaethylene glycol, septaethylene glycol, octaethylene glycol, propylene glycol, propylene glycol mono- and diesters of fats and fatty acids (e.g. propylene glycol monocaprylate, propylene glycol monolaurate), benzyl alcohol, glycerol, oleyl alcohol, mineral oil, lanolin/lanolin derivatives, petrolatum or other petroleum products suitable for application to the skin, propylene glycol mono- and diesters of fats and fatty acids, macrogols, macrogolglycerides or polyethylene glycol glycerides and fatty esters (e.g. stearoyl macrogolglycerides, oleoyl macrogolglycerides, lauroyl macrogolglycerides, linoleoyl macrogolglycerides), ethoxylated castor oil (e.g. Cremophor—a polyoxyl hydrogenated castor oil), C6-C30 triglycerides, natural oils, glucosides (e.g. cetearyl glucoside), surfactants, and mixtures thereof. More preferable the solubilizer is selected from diethylene glycol monoethyl ether (Transcutol®), oleyl alcohol, and mixtures thereof.

It is preferred that the formulations herein comprise from about 0.1% to about 99%, more preferably from about 1% to about 75%, by weight of solubilizer.

It is preferred that the formulations have a viscosity at 20° C. of from about 50 cps to about 50000 cps, more preferably from about 500 cps to about 40000 cps, even more preferably from about 5000 cps to about 30000 cps. Should the viscosity need to be adjusted it can be done by means of a viscosity modifying agent. Preferred viscosity modifiers are selected from polyethylene glycols, acrylic acid-based polymers (carbopol polymers or carbomers), polymers of acrylic acid crosslinked with allyl sucrose or allylpentaerythritol (carbopol homopolymers), polymers of acrylic acid modified by long chain (C10-C30) alkyl acrylates and crosslinked with allylpentaerythritol (carbopol copolymers), poloxamers also known as pluronics (block polymers; e.g. Poloxamer 124, 188, 237, 338, 407), waxes (paraffin, glyceryl monostearate, diethylene glycol monostearate, propylene glycol monostearate, ethylene glycol monosterate, glycol stearate), hard fats (e.g. Saturated C8-C18 fatty acid glycerides), xantham gum, polyvinyl alcohol, solid alcohols, and mixtures thereof.

In preferred embodiments the formulation contain one or more PEGs. It is preferred that the formulation comprises at least one PEG of average molecular weight about 2000 or less, preferably about 1500 or less, preferably about 1000 or less, preferably about 800 or less, preferably about 600 or less, preferably about 500 or less, preferably about 400 or less. It is preferred that the formulation comprises at least one PEG of average molecular weight about 3000 or more, preferably about 3350 or more, preferably about 3500 or more. It is preferred that the formulation comprises a mixture of PEG's. More preferably, one PEG has an average molecular weight of about 800 or less and one PEG has an average molecular weight of 3000 or more.

A preferred formulation for use in the present invention comprises photosensitizer (especially green-porphyrins), low molecular weight PEG such as PEG200, diethylene glycol monoethyl ether (Transcutol®), high molecular weight PEG such as PEG3350 and fatty alcohol such as oleyl alcohol.

The formulation herein may comprise a variety of other components. Any suitable ingredient may be used herein but typically these optional component will render the formulations more cosmetically acceptable or provide additional usage benefits. Some examples of preferred optional ingredients include, but are not limited to, emulsifiers, humectants, emollients, surfactants, oils, waxes, fatty alcohols, dispersants, skin-benefit agents, pH adjusters, dyes/colourants, analgesics, perfumes, preservatives, and mixtures thereof.

Examples of suitable preservatives include but are not limited to parabens, benzyl alcohol, quaternium 15, imidazolidyl urea, disodium EDTA, methylisothiazoline, alcohols, and mixtures thereof. Examples of suitable emulsifiers include but are not limited to waxes, sorbitan esters, polysorbates, ethoxylated castor oil, ethoxylated fatty alcohols, macrogolglycerides or polyethylene glycol glycerides and fatty esters (e.g. stearoyl macrogolglycerides, oleoyl macrogolglycerides, lauroyl macrogolglycerides), esters of saturated fatty acids (e.g. diethylene glycol parmitostearate), macrogols of cetostearyl ether (e.g. macrogol-6-cetostearyl ether), polymers of high molecular weight, crosslinked acrylic acid-based polymers (carbopols or carbomers), and mixtures thereof. Examples of suitable emollients include but are not limited to propylene glycol dipelargonate, 2-octyldodecyl myristate, non-polar esters, triglycerides and esters (animal and vegetable oils), lanolin, lanolin derivatives, cholesterol, glucosides (e.g. cetearyl glucoside), pegylated lanolin, ethoxylated glycerides, and mixtures thereof Examples of suitable surfactants include but are not limited to sorbitan esters, polysorbates, sarcosinates, taurate, ethoxylated castor oil, ethoxylated fatty alcohols, ethoxylated glycerides, caprylocaproyl macrogol-8 glycerides, polyglyceryl-6 dioleate, and mixtures thereof. Examples of suitable oils include but are not limited to propylene glycol monocaprylate, medium chain triglycerides (MCT), 2-octyl-dodecyl myristate, cetearyl ethylhexanoate, and mixtures thereof. Examples of suitable fatty alcohols include but are not limited to cetostearyl alcohol, cetyl alcohol, stearyl alcohol, and mixtures thereof. Also useful in the formulations herein are lipids and triglycerides (e.g. concentrates of Seed Oil Lipids, Concentrates of Marine Oil Lipids, high purity triglycerides and esters), alkyl ether sulfates, alkyl polyglycosides, alkylsulfates, amphoterics cream bases, and mixtures thereof.

Preparation of dry formulations that are reconstituted immediately before use also is contemplated. The preparation of dry or lyophilized formulations can be effected in a known manner, conveniently from the solutions of the invention. The dry formulations of this invention are also storable. By conventional techniques, a solution can be evaporated to dryness under mild conditions, especially after the addition of solvents for azeotropic removal of water, typically a mixture of toluene and ethanol. The residue is thereafter conveniently dried, e.g. for some hours in a drying oven.

The method herein is targeted to hair follicles and/or surrounding tissues and cells as a treatment for alopecia. The photosensitizer containing preparations of the invention may be administered systemically or locally and may be used alone or as components of mixtures. Preferably the administration is local. The route of administration for the photosensitizer may be topical, intradermal, intravenous, oral, or by use of an implant. Preferably the route of administration is topical. For example, green porphyrins may be administered by means including, but not limited to, topical lotions, topical creams, topical pastes, topical suspensions, intravenous injection or infusion, oral intake, or local administration in the form of intradermal injection or an implant. Additional routes of administration are subcutaneous, intramuscular, or intraperitoneal injections of the photosensitizers in conventional or convenient forms.

For topical formulations (such as ointments) to be applied to the surface of the skin, the concentration of the photosensitizer in the excipient preferably ranges from about 0.001 to about 10% w/w, and more preferably from about 0.005 to about 5% w/w, and even more preferably between about 0.01 to about 1% w/w. Particularly preferred is the use of about a 0.2% w/w topical formulation.

When administered topically, it is preferred that the area to be treated be massaged after application of the photosensitizer. While not wishing to be bound by theory, it is believed that the massage aids in the penetration and distribution of photosensitizer in the target tissue.

After administration, the photosensitizer will be present in hair follicles and the surrounding tissues and cells for photoactivation. Irradiation, with activation energy of appropriate wavelength and intensity, will be applied using an appropriate activation energy source, thereby activating the photosensitizer to stimulate and/or restore hair growth. Appropriate activation energy sources can be anything suitable. For example, sunlight or other ambient sources may be used but preferred for use are devices which allow a controlled energy dose to be delivered. By “stimulating” or “restoring” hair growth, all manner of inducing, activating, reviving, renewing, replacing or otherwise causing hair growth are included. Preferably, the irradiation is with visible light or comprises a wavelength of visible light.

Each photosensitizer requires activation with an appropriate wavelength(s) of radiation. As such, the methods of the invention may be conducted with any irradiation, preferably with light, which activates the photosensitizer used. Preferably, the irradiation contains one or more wavelength which is capable of penetrating the skin to activate the photosensitizer used. The wavelength(s) of radiation or light useful in the invention depends on the activation range of the photosensitizer used as part of the treatment method. Wavelengths of about 380-900 nanometers (nm) are preferred, depending upon the photosensitizer and upon the depth of tissue penetration desired. More preferred are wavelengths from about 400 to about 800 nm. For example, BPD-MA, a green porphyrin derivative, can be activated by red and blue light as well as ambient light containing wavelengths from 400-900 nm. Light having a wavelength shorter than 400 nm is acceptable, but not preferred because of the potentially damaging effects of UVA light.

Any appropriate activation energy source, depending on the absorption spectrum of the photosensitizer, may be used for photosensitizer activation. Preferred sources include, but are not limited to, lasers, light emitting diodes (LED), incandescent lamps, arc lamps, standard fluorescent lamps, U.V. lamps, and combinations thereof. More preferred are lasers, light emitting diodes, and combinations thereof

Alternatively any convenient source of activation energy having a component of wavelengths that are absorbed by the photosensitizer may be used, for example, an operating room lamp, or any bright light source, including sunlight. Wavelengths in the ultraviolet range should, however, generally be avoided because of their mutagenic potential. Therefore, it is preferred that the activation energy used for the methods herein is not in the ultraviolet range.

Commercially available activation energy sources include CureLight™ (available from Photocure ASA, Oslo, Norway), BLU-U™ (available from DUSA, Wilmington, Mass., USA), PDT Laser (available from Diomed, Andover, Mass., USA), Ceralas™ (available from Biolitec AG, Jena, Germany), and Q-Beam & Quanta-med (Quantum Devices Inc., Barneveld, Wis., USA).

The activation energy dose administered during the PDT treatment contemplated herein can vary as necessary. Preferably, for photosensitizers of high potency, such as green porphyrins, the dosage of the light is about 5-50 J/cm² for systemically-delivered drug and about 25-200 J/cm² for topically-delivered photosensitizers. It is generally preferred that the total dose of the irradiation should generally not exceed 200 J/cm², or more preferably not exceed 100 J/cm². Preferred doses can range between about 0.01 J/cm² to about 200 J/cm², more preferably 0.1 J/cm² to about 100 J/cm². For example, about 25, about 50, about 75, about 100, about 125, about 150, or about 175 J/cm². More preferred doses range from about 25 J/cm² to about 100 J/cm². Even more preferred doses range from about 40 J/cm² to about 80 J/cm², especially about 50 J/cm² to about 75 J/cm².

Normally, the intensity of the energy source should not exceed about 600-1000 mW/cm². Irradiances between about 10 and 400 mW/cm², and more preferably between 25 and 100 mW/cm².

Normally, the irradiation lasts from about 10 seconds to about 4 hours, and preferably between about 5 minutes and 1 hour. Irradiation times of about 10, about 15, about 20, about 30, about 45, about 60, about 75, about 90, about 105, about 120, about 135, about 150, about 165 and about 180 minutes may be used.

While not wishing to be bound by theory, it is believed that different photosensitizers, different formulations, and different activation energies will require different parameters in order to cause hair growth. Such parameters can be determined by simple dose-ranging studies. For example, a suitable method could involve:
(a) taking a terminal hair count,
(b) applying the photosensitizing composition at various strengths,
(c) waiting for varying lengths of time,
(d) treating with various activation energy doses
(e) reassessing terminal hair counts after a suitable interval.

Alternatively, the study might involve some other methods of assessing hair growth such as an assessment of the hair density, hair weight, and/or hair shaft diameter.

It is preferred that the present invention not involve a PDT dose that results in extensive cell death in the treatment area. PDT dose is determined by two factors, the amount of photosensitizer present and the amount of activation energy delivered. While not wishing to be bound by theory, it is believed that the mechanism by which PDT stimulates hair growth is through increasing the levels of pro-inflammatory cytokines. It is thought that these cytokines act through biochemical pathways to cause susceptible hair follicles to grow terminal hair. It is possible that there is a specific dose range where the PDT dose is high enough to increase the level of these pro-inflammatory cytokines but low enough to avoid unwarranted side-effects such as extensive cell death and the resultant tissue damage. In addition, as discussed above, it has been suggested that PDT could be used for hair removal and, while the inventors have not found that PDT aids hair removal and not wishing to be bound by theory, it is possible that at higher doses of PDT may affect the hair follicle in such a way that hair removal is aided while lower doses of PDT stimulates an increase in terminal hair numbers. As used herein, the term “low dose of PDT” refers to dose of PDT that don't result in extensive cell death.

It is preferred that the area to be treated have minimal hair coverage when the activation energy is applied. Therefore, if there is significant hair coverage of the area to be treated, it is preferred that the hair is cut short or shaved prior to activation energy application. While not wishing to be bound by theory, it is believed that, due to the fact that hair has a shielding function, hair coverage can affect the activation energy dose that is delivered to the target area, especially when visible light wavelengths are used. Consequently, in order to more accurately deliver the correct does it is preferred that there be little or no hair coverage. Alternatively, the shielding effect of the hair may be compensated for by changes to delivery of the activation energy.

The irradiation or light exposure used in the invention may be directed to a small or large area of the body or scalp depending on the patch to be treated. Treatment may be preceded with an assessment of the time of light exposure for the patient's minimal erythemal dose (MED) occurrence in order to avoid potential burning of the exposed skin.

The PDT may be a single treatment but it is preferred that the treatment is repeated. The frequency may vary. For example, the treatments could be daily, every two days, twice weekly, weekly, ever two weeks, twice monthly, every four weeks, monthly, every six weeks, every eight weeks, every two months, quarterly, twice annually, or annually, or other suitable time interval to stimulate hair growth or to maintain the prevailing condition. Preferably, the treatment is repeated at least once every six months. More preferably at least once every three months. Even more preferably at least once every two months.

The total number of treatments can range from one to as many as required. In cases where hair loss is observed, maintenance treatment on a regular basis may be initiated and sustained. It is preferred that the total number of treatments in any 3 month period be from 1 to 12, more preferably from 1 to 6, even more preferably from 2 to 3.

The time between administration of photosensitizer and administration of activation energy will vary depending on a number of factors. Activation energy delivery can take place at any suitable time following administration of photosensitizer as long as there is still photosensitizer present at the skin. Activation energy treatment within a period of about five minutes to about 6 hours after administration of the photosensitizer is preferred, with a range of 30 minutes to 4 hours being more preferred. Even more preferably the light is administered within a period of about 2 hours after administration of the photosensitizer.

Having now generally described the invention, the same will be more readily understood through reference to the following examples which are provided by way of illustration, and are not intended to be limiting of the present invention, unless specified.

### EXAMPLE

A total of 10 subjects were treated. All subjects were human males over 18 years of age and had type II or III Vertex alopecia, rated according to the modified Hamilton-Norwood scale.

All subjects received a single application of topical QLT0074 ointment 0.2% weight in weight (w/w) to two of three circular test sites on the vertex area of the scalp. The amount of ointment applied to each test site was approximately 224 mg (about 0.44mg of photosensitizer per test site). After 2 hours the excess drug was removed and red light (LED's—690nm) was administered to two of the three sites. The other test site served as a control with no drug or light administration.

Two light dose cohorts (50 and 75 J/cm²) were investigated with each cohort having 5 subjects.

Safety was assessed by monitoring all adverse events during and after the treatment. No serious adverse events were reported.

The efficacy was assessed by hair counts 3 months after the treatment and compared to baseline counts. These results are shown in Table 1:

TABLE 1

| | 50 J/cm² | 75 J/cm² |
|---|---|---|
| | (% change in terminal hair | (% change in terminal hair |
| | count) | count) |
| Drug | +8% | +4% |

| | |
|---|---|
| Control | −1.7% |

These results show that a single PDT treatment can produce an increase in hair count in subjects having AGA associated hair loss.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without undue experimentation. This application is intended to cover any variations, uses, or adaptations of the invention, following in general the principles of the invention, that include such departures from the present disclosure as come within known or customary practice within the art to which the invention pertains and as may be applied to the essential features hereinbefore set forth.

1. A method to increase the number of terminal hairs on the skin of a subject in need of such increase comprising:
(a) administering photosensitizer to an area of skin of said subject where hair growth is desired; and
(b) irradiating the area with energy containing a wavelength appropriate to activate the photo sensitizer;
wherein the number of terminal hairs in the treated area is increased.

2. The method of claim 1, wherein the irradiating delivers a low dose of PDT.

3. The method of claim 1, wherein increase in the number of terminal hairs in the treated area is at least 2% within 3 months.

4. The method of claim 2, wherein the increase in the number of terminal hairs in the treated area is at least 2% within 3 months.

5. The method of claim 1, wherein administering in step (a) is topical.

6. The method of claim 5, wherein said topical administrating includes massaging on the area where hair growth is desired.

7. The method of claim 5, wherein the photosensitizer is administered in a topical formulation having a viscosity at 20° C. of from about 50 cps to about 50000 cps.

8. The method of claim 1, wherein the photosensitizer is selected from the group consisting of pro-porphyrins, porphyrins, porphyrin derivatives, and mixtures thereof.

9. The method of claim 1, wherein the irradiating of step (b) is conducted by delivering said energy at a time 5 minutes to 4 hours after administering the photosensitizer.

10. The method of claim 1, wherein the total dose of energy in step (b) does not exceed 200 J/cm².

11. The method of claim 1, which further includes treating the subject with at least one non-photodynamic treatment that causes an increase in the number of terminal hairs within the treatment area.

12. The method of claim 1, wherein the subject is treated with at least one non-photodynamic treatment is selected from the group consisting of 5-alpha reductase inhibitors, minoxidil, hair transplantation, scalp reduction, and combinations thereof.

13. The method of claim 1, wherein said the level of at least one pro-inflammatory cytokine in the treated area is increased.

14. The method of claim 13, wherein the at least one pro-inflammatory cytokine is selected from the group consisting of granulocyte-macrophage colony stimulating factor, interleukin-1-α, and interleukin-1-β.

15. The method of claim 1, wherein the subject is suffering from hair loss caused by androgenetic alopecia.

16. A method of determining any increase in hair growth in a first area of skin exhibiting hair growth reduction or hair loss, said method comprising:
(a) administering a photosensitizer to said first area of said skin;
(b) irradiating said first area with electromagnetic energy containing a wavelength absorbed by said photosensitizer to activate it; and
(c) comparing the hair growth, in said first area to hair growth in a second area of said skin that has not been treated with both (a) and (b)
whereby any increase in hair growth in said first area is determined.

17. The method of claim 16, wherein said photosensitizer absorbs radiation in the range of 400 nm to 800 nm.

18. The method of claim 16, wherein the photosensitizer is administered by topical application.

19. The method of claim 16, wherein said energy is visible light.

20. The method of claim 16, wherein the in hair growth is measured by counting the number of terminal hairs, measuring hair weight, measuring hair density, and/or measuring hair shaft diameter.
